(19) United States
(12) Patent Application Publication
(10) Pub. No.: US 2019/0053890 A1
(43) Pub. Date: Feb. 21, 2019

(54) AZOBENZENE GEL AND METHOD FOR MANUFACTURING SAME AND ARTIFICIAL INTRAOCULAR LENS DEVICE

(71) Applicant: HON HAI PRECISION INDUSTRY CO., LTD., New Taipei (TW)

(72) Inventor: HSIU-WEN CHIEN, New Taipei (TW)

(21) Appl. No.: 15/825,118

(22) Filed: Nov. 29, 2017

(30) Foreign Application Priority Data
Aug. 18, 2017 (TW) 106128185

(51) Int. Cl. A61F 2/16; C08L 33/08; C08L 83/04; C07C 245/08; C07C 69/54

(52) U.S. Cl. CPC A61F 2/16; C08L 33/08; C08L 83/04; A61F 2002/16965; C07C 69/54; C08L 2201/54; C08L 2203/02; C07C 245/08

(57) ABSTRACT

An artificial intraocular lens device with light-induced zoom-in and zoom-out properties includes a lens, an inert lens frame surrounding the lens; and two inert supporting arms formed at the side edge of the lens frame. The lens is made from azobenzene gel, the deformation of the azobenzene gel by expansion or contraction respectively reverses on exposure to visible light and to ultraviolet light. The azobenzene gel is formed by heating an azobenzene gel precursor for polymerization, the azobenzene gel precursor being composed of monomer, initiator, crosslinker, and azobenzene.

### FIELD

The subject matter herein generally relates to the field of intraocular lenses (IOL) and, more particularly, to azobenzene gel, and method for manufacturing same and artificial intraocular lens device to automatically zoom.

### BACKGROUND

The human eye provides vision by transmitting light through the clear cornea, and focusing the image by way of a crystalline lens onto a retina. The quality of the focused image depends on many factors including the size and shape of the eye, and the transparency of the cornea and the crystalline lens.

When age or disease causes the crystalline lens to become less transparent, vision deteriorates because of the diminished light which can be transmitted to the retina. One such deficiency is medically known as a cataract. An accepted treatment for this condition is surgical removal, and replacing of the crystalline lens with an intraocular lens (IOL) which is artificial. However, the traditional artificial IOL has a fixed focus, and it is difficult to provide zoom.

Therefore, it is desirable to provide an azobenzene gel and method for manufacturing same and an artificial IOL device which can overcome the above-mentioned disadvantages.

### BRIEF DESCRIPTION OF THE DRAWINGS

Implementations of the present technology will now be described, by way of example only, with reference to the attached figures.

FIG. 1 is a plan view of an artificial intraocular lens device in accordance with one exemplary embodiment.

FIG. 2 is a side view of the artificial IOL device in FIG. 1.

FIG. 3 is a view when incident ultraviolet light strikes the artificial IOL device.

FIG. 4 is a view of the artificial IOL device recovered to its previous state.

FIG. 5 is a flowchart of a method for manufacturing azobenzene gel.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better. The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

Several definitions that apply throughout this disclosure will now be presented.

The term “substantially” is defined to be essentially conforming to the particular dimension, shape, or other feature that the term modifies, such that the component need not be exact. For example, “substantially cylindrical” means that the object resembles a cylinder, but can have one or more deviations from a true cylinder. The term “comprising,” when utilized, means “including, but not necessarily limited to”; it specifically indicates open-ended inclusion or membership in the so-described combination, group, series, and the like. The references “a plurality of” and “a number of” mean “at least two.”

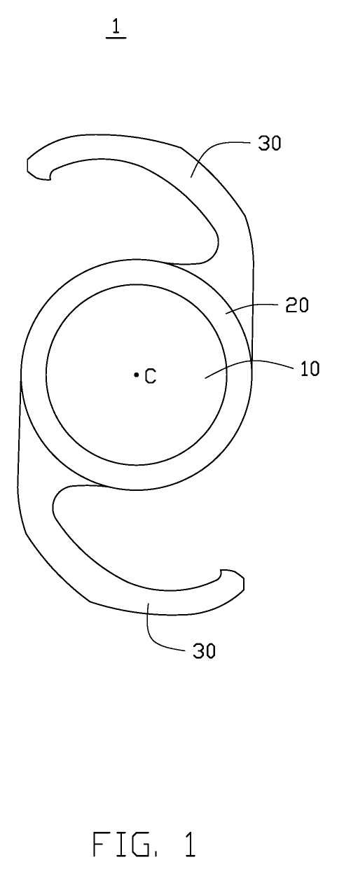

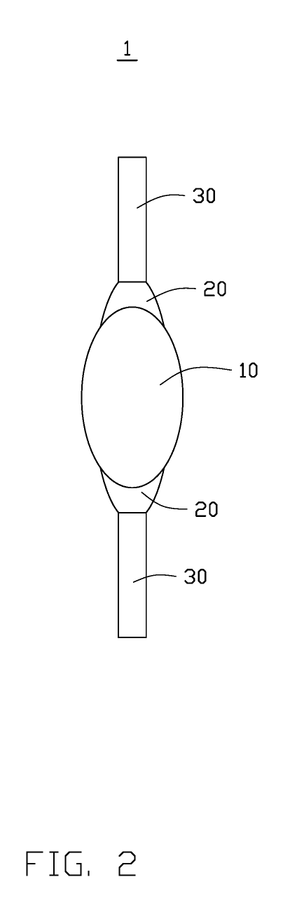

FIGS. 1-4 illustrate an artificial intraocular lens (AIOL) device 100 according to one embodiment. The AIOL device 100 includes a lens 10, a lens frame 20 surrounding the lens 10 and two supporting arms 30 formed at the side edge of the lens frame 20. The two supporting arms 30 are opposite to each other and are substantially open-loops. A diameter of the lens 10 is about 4˜6 millimeters. A diameter of the lens frame 20 is about 5˜6.5 millimeters. A length of the AIOL device 100 is about 11˜13 millimeters.

The lens 10 is made from azobenzene gel. In this embodiment, the azobenzene gel is formed by heating azobenzene gel precursor for polymerization. The azobenzene gel precursor is composed of monomer, initiator, crosslinker, and azobenzene.

The monomer is hydrophilic monomer or silicone monomer.

Preferably, the hydrophilic monomer is acrylic (CR′H═CRCOX) monomer, R in this chemical formula is H or CH3, R′ in this chemical formula is selected from H, CH3, or alkyl; X in this chemical formula is O, N or hydrophilic groups. More preferably, the hydrophilic monomer is selected from a group consisting of 2-hydroxyethyl methacrylate (HEMA), N-Dimethylacrylamide (DMA), methyl methacrylate (MAA), N-vinyl pyrrolidone (NVP), polyethylene glycol methacrylate (PEGMA), and sulfobetaine methacrylate (SBMA) or any combination thereof.

The silicon monomer can be trihydroxymethyl aminomethane (TRIS), or polydimethylsiloxane (PDMS) or combination of trihydroxymethyl aminomethane and polydimethylsiloxane.

Preferably, A weight ratio of the monomer in the azobenzene gel precursor is in the range from about 50%-98%.

Preferably, A weight ratio of the initiator in the azobenzene gel precursor is in the range from about 0.03%-16.2%.

The crosslinker is select from a group consisting of ethylene glycol dimethacrylate (EGDMA), trimethylolpropane trimethacrylate (TMPTMA), N, N′-methylene bis (MBAA) or any combination thereof. A weight ratio of the crosslinker in the azobenzene gel precursor is in the range from about 0.01%-13.6%.

The azobenzene is 4, 4′-bis[6-(acryloyloxy)hexyloxy]azobenzene. The amolecular formula of the azobenzene is:

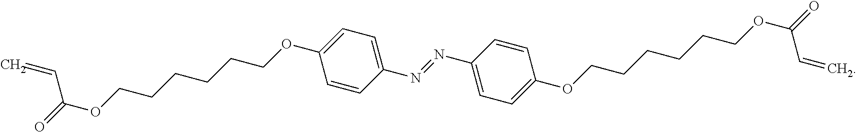 [SMILES: C=CC(=O)OCCCCCCOc1ccc(/N=N/c2ccc(OCCCCCCOC(=O)C=C)cc2)cc1]

A weight ratio of the azobenzene in the azobenzene gel precursor is in the range from about 1.1%-21.8%. Azobenzene is a modified group as gel, and the azobenzene undergoes a trans- and cis-configuration changes under different wavelengths when exposed to UV light. The trans- and cis-configuration reaction is:

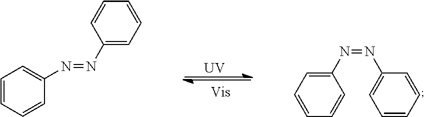 [SMILES: c1ccc(/N=N/c2ccccc2)cc1 ; "UV" ; c1ccc(/N=N\c2ccccc2)cc1]

wherein “UV” condition in the reaction represents the azobenzene gel exposed to UV light; and “Vis” condition in the reaction represents the azobenzene gel exposed to visible light. The azobenzene gel reaction is reversible when exposed to UV light and visible light.

The lens frame 20 and the two supporting arms 30 are made from hydrogel or silicone hydrogel. That is, the lens frame 20 the two supporting arms 30 are not affected when exposed to UV light.

The lens 10 is made from azobenzene gel, thereby, the azobenzene gel is light reversible. When the AIOL device 100 is worn and is exposed to UV light, the AIOL device 100 expands or contracts in response to the UV light. The lens frame 20 and the two supporting arms 30 do not respond to the UV light, that is, the lens frame 20 and the two supporting arms 30 do not deform when exposed to UV light. Thus the lens frame 20 and the two supporting arms 30 merely fix the lens 10 in place, the expansion and contraction of the lens 10 occur along a direction perpendicular to an extension direction of the two supporting arms 30. Thereby, focal length changes are realized, to change the focal length of the AIOL device 100.

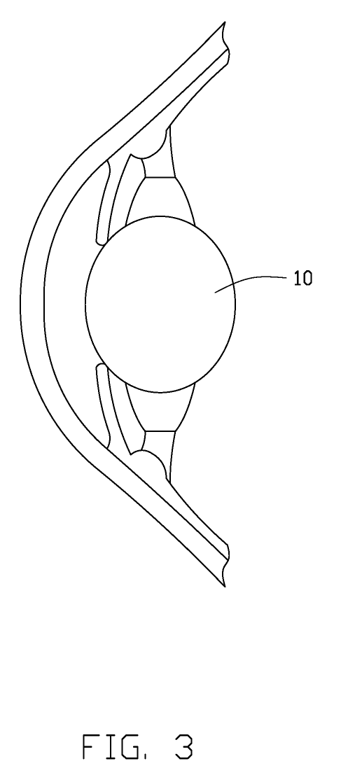

Specifically, as shown in FIG. 3, when incident ultraviolet light strikes the lens 10, the lens 10 expands. The frame 20 and the two supporting arms 30 hold steady, and the expansion of the lens 10 occurs along a direction perpendicular to a extension direction of the two supporting arms 30, thereby auto-zooming is realized.

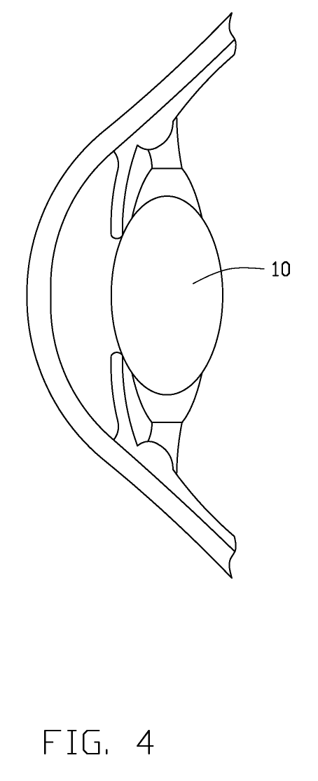

As shown in FIG. 4, when the AIOL device 100 is exposed to visible light, the lens 10 contracts. The contraction of the lens 10 occurs along a direction perpendicular to a extension direction of the two supporting arms 30, thereby auto-zooming is realized.

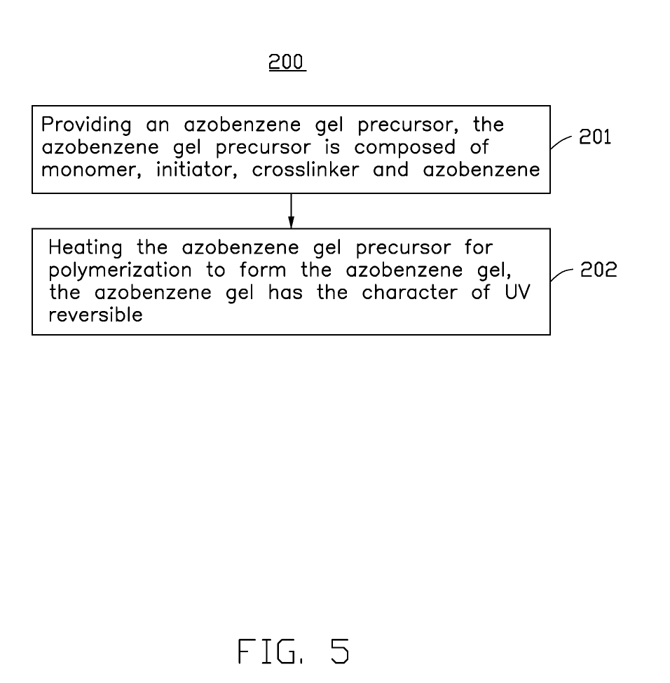

FIG. 5 illustrates a method 200 for manufacturing an azobenzene gel according to one embodiment. The method is provided by way of example as there are a variety of ways to carry out the method. The method can begin at block 201.

At block 201, an azobenzene gel precursor is provided. The azobenzene gel precursor comprises of monomer, initiator, crosslinker and azobenzene.

The monomer is hydrophilic monomer or silicone monomer.

Preferably, the hydrophilic monomer is acrylic (CR′H═CRCOX) monomer, R in this chemical formula is H or CH3; R′ in this chemical formula is selected from H, CH3 or alkly, X in this chemical formula is O, N or hydrophilic groups. More preferably, the hydrophilic monomer is select from a group consisting of 2-hydroxyethyl methacrylate (HEMA), N-Dimethylacrylamide (DMA), methyl methacrylate (MAA), N-vinyl pyrrolidone (NVP), polyethylene glycol methacrylate (PEGMA) and sulfobetaine methacrylate (SBMA) or any combination thereof.

The silicon monomer can be trihydroxymethyl aminomethane (TRIS) or polydimethylsiloxane (PDMS), or combination of trihydroxymethyl aminomethane and polydimethylsiloxane.

Preferably, a weight ratio of hydrophilic monomer in the azobenzene gel precursor is in the range from about 50%-98%.

Preferably, a weight ratio of initiator in the azobenzene gel precursor is in the range from about 0.03%-16.2%.

The crosslinker is select from a group consisting of ethylene glycol dimethacrylate (EGDMA), trimethylolpropane trimethacrylate (TMPTMA), N, N′-methylene bis (MBAA) or any combination thereof. A weight ratio of crosslinker in the azobenzene gel precursor is in the range from about 0.01%-13.6%.

The azobenzene is 4,4′-bis[6-(acryloyloxy)hexyloxy]azobenzene. And the molecular formula is:

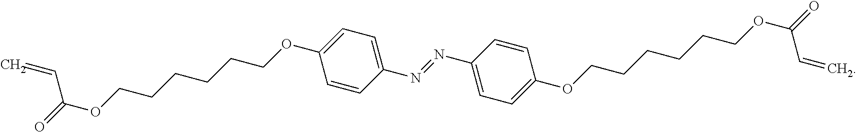 [SMILES: C=CC(=O)OCCCCCCOc1ccc(/N=N/c2ccc(OCCCCCCOC(=O)C=C)cc2)cc1]

A weight ratio of the azobenzene in the azobenzene gel precursor is in the range from about 1.1%-21.8%.

At block 202, the azobenzene gel precursor is heated for polymerization to form the azobenzene gel. And the azobenzene gel is able to form the AIOL device 100.

The embodiments shown and described above are only examples. Therefore, many commonly-known features and details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size, and arrangement of the parts within the principles of the present disclosure, up to and including the full extent established by the broad general meaning of the terms used in the claims. It will, therefore, be appreciated that the embodiments described above may be modified within the scope of the claims.

What is claimed is:

1. An azobenzene gel comprising:
the azobenzene gel has the character of UV reversible, the azobenzene gel is formed by heated an azobenzene gel precursor for polymerization, the azobenzene gel precursor is composed of monomer, initiator, crosslinker and azobenzene.

2. The azobenzene gel of claim 1, wherein the azobenzene is 4,4′-bis[6-(acryloyloxy)hexyloxy]azobenzene, and a molecular formula of the azobenzene is as follow:

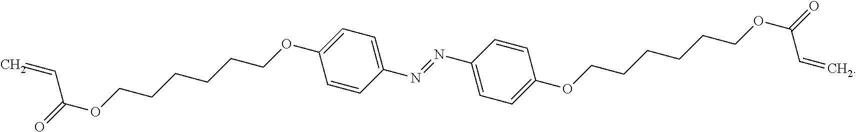 [SMILES: C=CC(=O)OCCCCCCOc1ccc(/N=N/c2ccc(OCCCCCCOC(=O)C=C)cc2)cc1]

3. The azobenzene gel of claim 2, wherein a weight ratio of azobenzene in the azobenzene gel precursor is in the range from 1.1%-21.8%.

4. The azobenzene gel of claim 3, wherein the monomer is hydrophilic monomer or silicone monomer.

5. The azobenzene gel of claim 4, wherein a weight ratio of the monomer in the azobenzene gel precursor is in the range from 50%-98%.

6. The azobenzene gel of claim 1, wherein a weight ratio of initiator in the azobenzene gel precursor is in the range from 0.03%-16.2%.

7. The azobenzene gel of claim 2, wherein the crosslinker is selected from a group consisting of ethylene glycol dimethacrylate, trimethylolpropane trimethacrylate, N, N′-methylene bis or any combination thereof.

8. The azobenzene gel of claim 7, wherein a weight ratio of the crosslinker in the azobenzene gel precursor is in the range from about 0.01%-13.6%.

9. A method for manufacturing an azobenzene gel comprising:
providing an azobenzene gel precursor, the azobenzene gel precursor comprising of monomer, initiator, crosslinker and azobenzene; and
heating the azobenzene gel precursor for polymerization to form the azobenzene gel, the azobenzene gel has the character of UV reversible.

10. The method of claim 9, wherein the azobenzene is 4,4′-bis[6-(acryloyloxy)hexyloxy]azobenzene, and a molecular formula of the azobenzene is as follow:

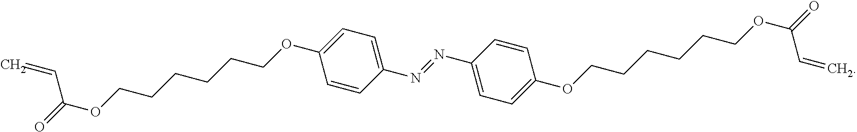 [SMILES: C=CC(=O)OCCCCCCOc1ccc(/N=N/c2ccc(OCCCCCCOC(=O)C=C)cc2)cc1]

11. The method of claim 10, wherein a weight ratio of the azobenzene in the azobenzene gel precursor is in the range from about 1.1%-21.8%.

12. The method of claim 9, wherein the monomer is hydrophilic monomer or silicone monomer.

13. The method of claim 12, wherein a weight ratio of the monomer in the azobenzene gel precursor is in the range from 50%-98%.

14. The method of claim 9, wherein a weight ratio of initiator in the azobenzene gel precursor is in the range from 0.03%-16.2%.

15. The method of claim 9, wherein the crosslinker is select from a group consisting of ethylene glycol dimethacrylate, trimethylolpropane trimethacrylate, N, N′-methylene bis or any combination thereof.

16. The method of claim 15, wherein a weight ratio of the crosslinker in the azobenzene gel precursor is in the range from about 0.01%-13.6%.

17. An artificial intraocular lens device, comprising:
a lens;
a lens frame surrounding the lens; and
two supporting arms formed at the side edge of the lens frame; wherein the lens is made from azobenzene gel, the azobenzene gel has the character of UV reversible, the azobenzene gel is formed by heated an azobenzene gel precursor for polymerization, the azobenzene gel precursor is composed of monomer, initiator, crosslinker and azobenzene.

18. The artificial intraocular lens device of claim 17, wherein the lens frame and the two supporting arms are made from hydrogel or silicone hydrogel.

19. The artificial intraocular lens device of claim 18, wherein a diameter of the lens is about 4˜6 millimeters; a diameter of the lens frame is about 5˜6.5 millimeters, a length of the artificial intraocular lens is about 11˜13 millimeters.
